(19) United States
(12) Patent Application Publication
(10) Pub. No.: US 2019/0223544 A1
(43) Pub. Date: Jul. 25, 2019

(54) CIRCULATION THERAPY ASSEMBLY

(71) Applicant: Richard Strong, Florissant, MO (US)

(72) Inventor: Richard Strong, Florissant, MO (US)

(21) Appl. No.: 15/878,726

(22) Filed: Jan. 24, 2018

(51) Int. Cl. A43B 7/04; A43B 3/02; A61F 7/00; H05B 3/42; A43B 3/00; H01M 10/42

(52) U.S. Cl. CPC A43B 7/04; A43B 3/02; A61F 7/007; H05B 3/42; A61F 2007/0078; A61F 2007/0039; A61F 2007/0086; H01M 10/425; H01M 2220/30; A43B 3/0005

(57) ABSTRACT

A circulation therapy assembly includes a pair of boots that is each of the boots worn on one of a pair of legs. Each of the boots is comprised of a thermally insulating material and each of the boots has a height of at least 55.0 cm. Thus, each of the boots extends above a knee on the legs when the boots are worn. A pair of inserts is provided and each of the inserts is removably positioned within an associated one of the boots. Each of the inserts surrounds the associated leg and a corresponding foot when the boots are worn. Moreover, each of the inserts is comprised of a thermally conductive material. A pair of heating units is each coupled to an associated one of the inserts and each of the heating units heats the associated insert when the heating units is turned on. In this way each of the heating units heats the associated leg when the boots are worn thereby enhancing blood circulation in the associated leg.

### CROSS-REFERENCE TO RELATED APPLICATIONS

### STATEMENT REGARDING FEDERALLY SPONSORED RESEARCH OR DEVELOPMENT

### Not Applicable

### THE NAMES OF THE PARTIES TO A JOINT RESEARCH AGREEMENT

### Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

### Not Applicable

### STATEMENT REGARDING PRIOR DISCLOSURES BY THE INVENTOR OR JOINT INVENTOR

### Not Applicable

### BACKGROUND OF THE INVENTION

### (1) Field of the Invention

### (2) Description of Related Art Including Information Disclosed Under 37 CFR 1.97 and 1.98

The disclosure and prior art relates to therapy devices and more particularly pertains to a new therapy device for PURPOSE.

### BRIEF SUMMARY OF THE INVENTION

An embodiment of the disclosure meets the needs presented above by generally comprising a pair of boots that is each of the boots worn on one of a pair of legs. Each of the boots is comprised of a thermally insulating material and each of the boots has a height of at least 55.0 cm. Thus, each of the boots extends above a knee on the legs when the boots are worn. A pair of inserts is provided and each of the inserts is removably positioned within an associated one of the boots. Each of the inserts surrounds the associated leg and a corresponding foot when the boots are worn. Moreover, each of the inserts is comprised of a thermally conductive material. A pair of heating units is each coupled to an associated one of the inserts and each of the heating units heats the associated insert when the heating units is turned on. In this way each of the heating units heats the associated leg when the boots are worn thereby enhancing blood circulation in the associated leg.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a front view of a circulation therapy assembly according to an embodiment of the disclosure.

FIG. 2 is a back view of an embodiment of the disclosure.

FIG. 3 is a front perspective view of an embodiment of the disclosure.

FIG. 4 is a right view of an embodiment of the disclosure.

FIG. 5 is a bottom view of an embodiment of the disclosure.

FIG. 6 is a cross sectional view taken along line 6-6 of FIG. 5 of an embodiment of the disclosure.

FIG. 7 is a perspective in-use view of an embodiment of the disclosure.

### DETAILED DESCRIPTION OF THE INVENTION

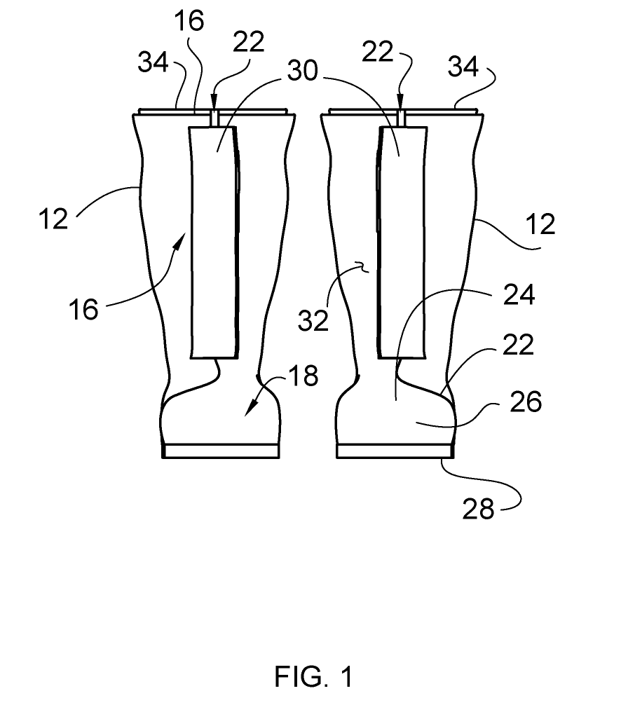

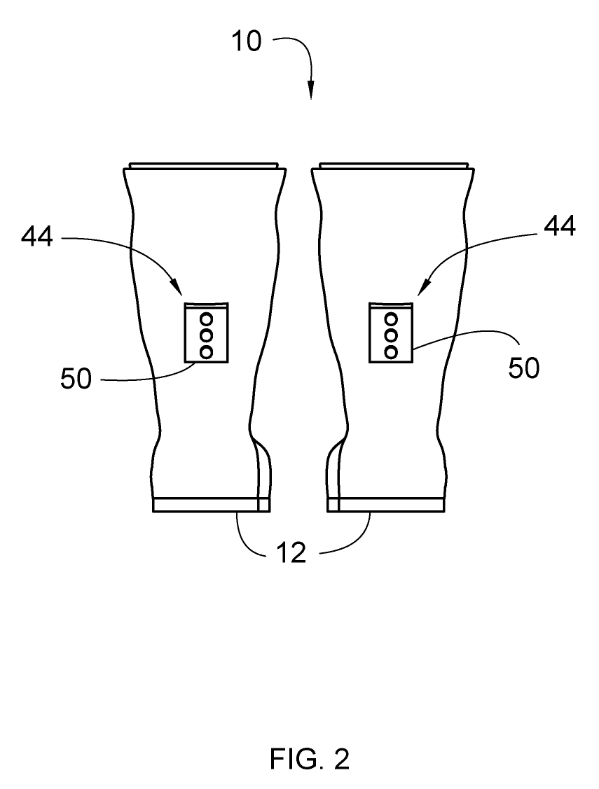

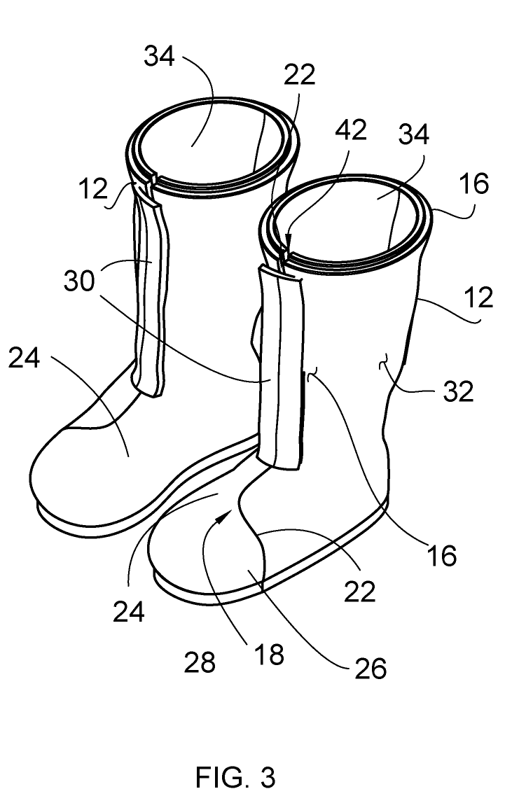

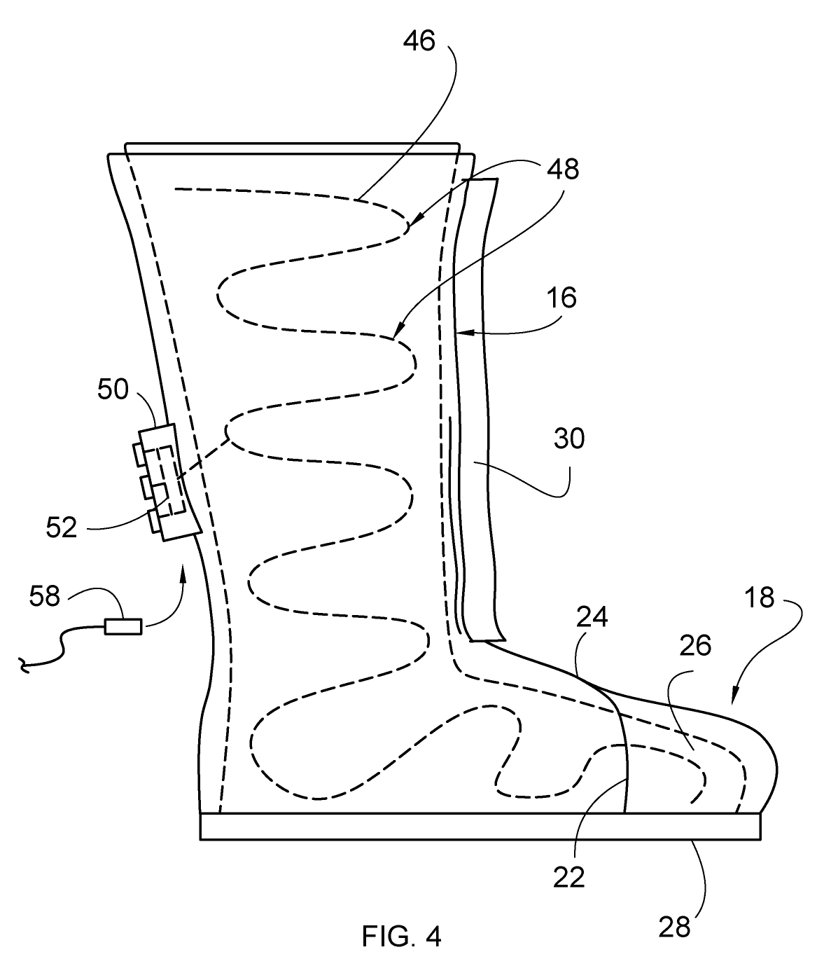

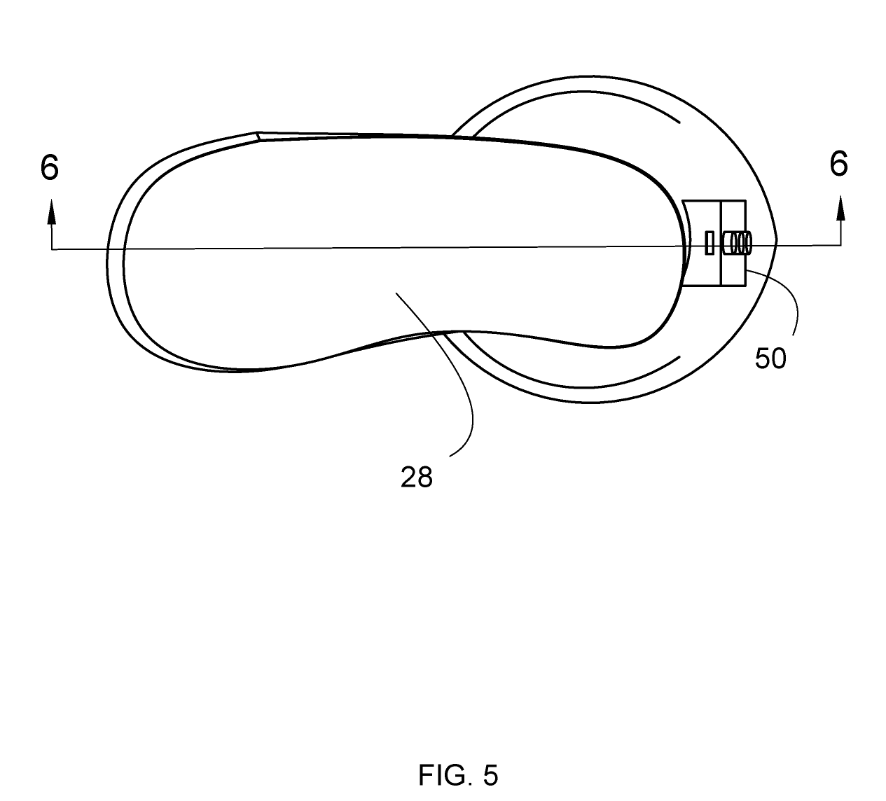

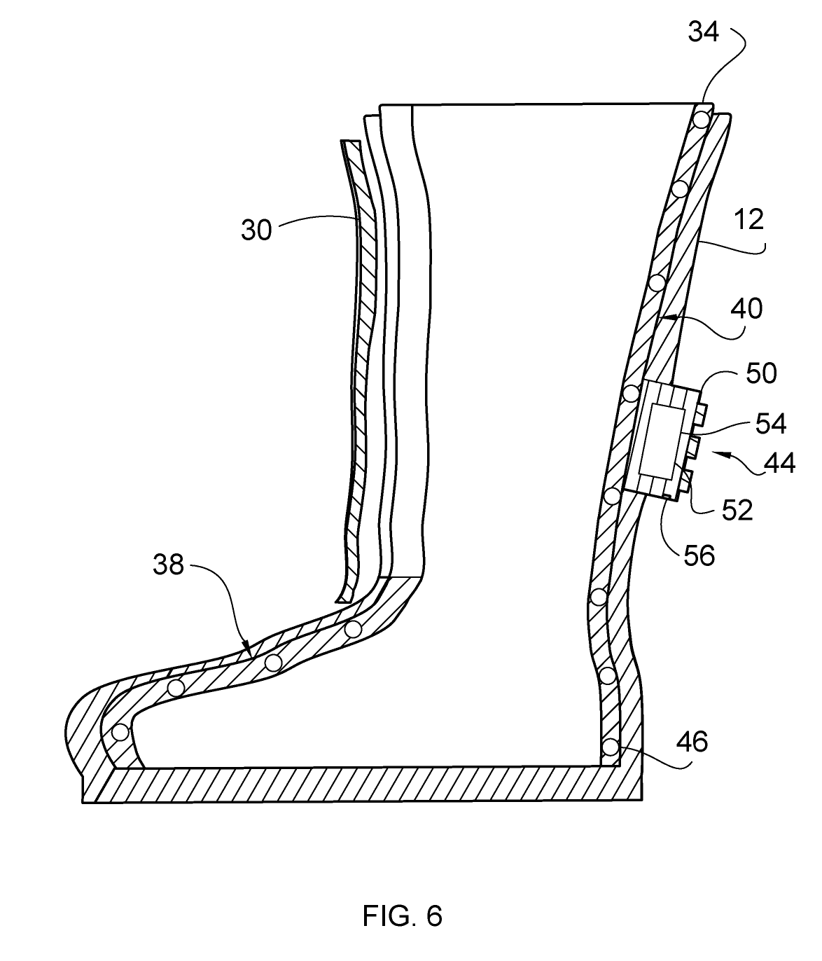

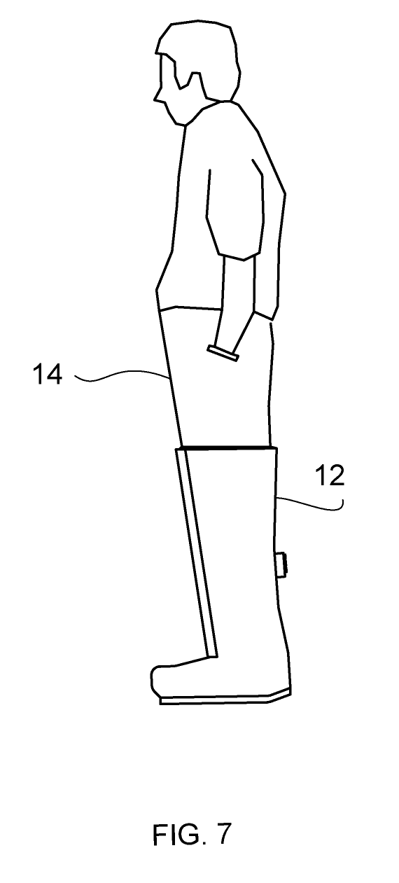

With reference now to the drawings, and in particular to FIGS. 1 through 7 thereof, a new therapy device embodying the principles and concepts of an embodiment of the disclosure and generally designated by the reference numeral 10 will be described.

As best illustrated in FIGS. 1 through 7, the circulation therapy assembly 10 generally comprises a pair of boots 12 that is each worn on one of a pair of legs 14. Each of the boots 12 is comprised of a thermally insulating material such as leather or the like. Moreover, each of the boots 12 has a height of at least 55.0 cm to extend above a knee on the legs 14. Each of the boots 12 has a front quarter 16 and a vamp 18, and the front quarter 16 has a top edge 20. Additionally, each of the boots 12 has a cut 22 extending through the front quarter 16 and the vamp 18. The cut 22 on each of the boots 12 extends between the top edge 20 and a top side 24 of the vamp 18. Moreover, the cut 22 on each of the boots 12 curves downwardly along a lateral side 26 of the vamp 18 toward a sole 28 of the corresponding boot 12.

A pair of flaps 30 is each attached to an associated one of the boots 12. Each of the flaps 30 is positioned on the front quarter 16 of the associated boot 12. Additionally, each of the flaps 30 is oriented co-linear with the cut 22 responding to the front quarter 16 of the associated boot 12. Each of the flaps 30 releasably engages an outer surface 32 of the associated boot 12 to close the cut 22 in the associated boot 12. In this way each of the flaps 30 tightens and retains the associated boot 12 on a variety of sizes of legs 14. Each of the flaps 30 may include a mating member, such as a hook and loop fastener or the like, and the mating member may releasably engage the boot at a chosen point.

A pair of inserts 34 is provided and each of the inserts 34 is removably positioned within an associated one of the boots 12. Thus, each of the inserts 34 surrounds the associated leg 14 and a corresponding foot of the leg 14 when the boots 12 are worn. Each of the inserts 34 has a foot portion 38 and a leg portion 40 and each of the inserts 34 is comprised of a thermally conductive material. Each of the inserts 34 may have a cut 42 extending through the foot portion 38 and the leg portion 40. Additionally, the cut 42 in each of the inserts 34 may be aligned with the cut 22 in the associated boot 12.

A pair of heating units 44 is provided and each of the heating units 44 is coupled to an associated one of the inserts 34. Each of the heating units 44 is in thermal communication with the associated insert 34 and each of the heating units 44 heats the associated insert 34 when the heating units 44 are turned on. In this way each of the heating units 44 heats the associated leg 14 when the boots 12 are worn thereby enhancing blood circulation in the associated leg 14. Thus, each of the heating units 44 facilitates therapeutic benefits similar to that of soaking the legs 14 in heated water.

Each of the heating units 44 comprises a heating element 46 that is positioned within the associated insert 34. The heating element 46 is in thermal communication with the associated insert 34. Moreover, the heating element 46 generates heat when the heating element 46 is turned on to heat the associated insert 34. In this way the heating element 46 heats the associated leg 14. The heating element 46 is looped repeatedly to form a plurality of coils 48 and the coils 48 are distributed along the foot portion 38 and the leg portion 40 of the associated insert 34. The heating element 46 may be an electric heating element 46 with an operational temperature ranging between approximately 100.0 degrees Fahrenheit and 115.0 degrees Fahrenheit.

Each of the heating units 44 includes a control 50 that is coupled to the associated insert 34. The control 50 is electrically coupled to the heating element 46 and the control 50 turns the heating element 46 on and off. Moreover, the control 50 actuates the heating element 46 between a minimum temperature, a middle temperature and a maximum temperature. The control 50 may include a plurality of buttons for selecting the minimum, middle and maximum temperatures along with an on/off button. Additionally, the control 50 may extend outwardly through the corresponding boot 12 so the control 50 is accessible when the corresponding boot 12 is worn.

A power supply 52 is coupled to the control 50 and the power supply 52 is in electrical communication with the control 50. The power supply 52 comprises at least one battery 54 and a charge port 56 that is coupled to the control 50. The charge port 56 is electrically coupled to the at least one battery 54 and the charge port 56 is electrically coupled to a power source 58 for charging the at least one battery 54. The power source 58 may be a battery 54 charger or the like.

In use, each of the boots 12 is worn to provide heat therapy for a variety of leg ailments, such as poor circulation, muscle cramps, nerve pain and any other leg related ailment. The cut 22 in each of the boots 12 is spread apart to position the boots around the associated leg. The flap 30 on each boot 12 is pulled across the cut 22 to close the boot 12 around the assoicated leg 14. The control 50 is manipulated to turn the heating element 46 in each of the inserts 34 on and to choose the minimum, medium and maximum temperature. In this way the entire length of the leg 14 below the knee as well as the foot 36 is heated for the heat therapy. A user may engage in limited activity when the boots 12 are being worn for heat therapy. In this way the user remains mobile during the heat therapy unlike when the user soaks in heated water for the heat therapy.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A circulation therapy assembly being configured to be worn on legs thereby enhancing circulation in the legs, said assembly comprising:
a pair of boots, each of said boots being configured to be worn on one of a pair of legs, each of said boots being comprised of a thermally insulating material, each of said boots having a height of at least 55.0 cm wherein each of said boots is configured to extend above a knee on the legs;
a pair of inserts, each of said inserts being removably positioned within an associated one of said boots wherein each of said inserts is configured to surround the associated leg and a corresponding foot when said boots are worn, each of said inserts being comprised of a thermally conductive material; and
a pair of heating units, each of said heating units being coupled to an associated one of said inserts, each of said heating units being in thermal communication with said associated insert, each of said heating units heating said associated insert when said heating units is turned on wherein each of said heating units is configured to heat the associated leg when said boots are worn thereby enhancing blood circulation in the associated leg.

2. The assembly according to claim 1, wherein each of said boots has a front quarter and a vamp, said front quarter having a top edge, each of said boots having a cut extending through said front quarter and said vamp, said cut on each of said boots extending between said top edge and a top side of said vamp, said cut on each of said boots curving downwardly along a lateral side of said vamp toward a sole of said corresponding boot.

3. The assembly according to claim 2, further comprising a pair of flaps, each of said flaps being attached to an associated one of said boots, each of said flaps being positioned on said front quarter of said associated boot, each of said flaps being oriented co-linear with said cut responding to said front quarter of said associated boot, each of said flaps releasably engaging an outer surface of said associated boot to close said cut in said associated boot wherein each of said flaps is configured to retain said associated boot on the leg.

4. The assembly according to claim 1, wherein:
each of said inserts has a foot portion and a leg portion; and
each of said heating units comprises a heating element being positioned within said associated insert, said heating element being in thermal communication with said associated insert, said heating element generating heat when said heating element is turned on to heat said associated insert, said heating element being looped repeatedly to form a plurality of coils, said coils being distributed along said foot portion and said leg portion of said associated insert.

5. The assembly according to claim 4, further comprising a control being coupled to said associated insert, said control being electrically coupled to said heating element such that said control turns said heating element on and off, said control actuating said heating element between a minimum temperature, a middle temperature and a maximum temperature.

6. The assembly according to claim 5, further comprising a power supply being coupled to said control, said power supply being in electrical communication with said control.

7. The assembly according to claim 6, wherein said power supply comprises:
at least one battery; and
a charge port being coupled to said control, said charge port being electrically coupled to said at least one battery, said charge port being configured to be electrically coupled to a power source for charging said at least one battery.

8. A circulation therapy assembly being configured to be worn on legs thereby enhancing circulation in the legs, said assembly comprising:
a pair of boots, each of said boots being configured to be worn on one of a pair of legs, each of said boots being comprised of a thermally insulating material, each of said boots having a height of at least 55.0 cm wherein each of said boots is configured to extend above a knee on the legs, each of said boots having a front quarter and a vamp, said front quarter having a top edge, each of said boots having a cut extending through said front quarter and said vamp, said cut on each of said boots extending between said top edge and a top side of said vamp, said cut on each of said boots curving downwardly along a lateral side of said vamp toward a sole of said corresponding boot;
a pair of flaps, each of said flaps being attached to an associated one of said boots, each of said flaps being positioned on said front quarter of said associated boot, each of said flaps being oriented co-linear with said cut responding to said front quarter of said associated boot, each of said flaps releasably engaging an outer surface of said associated boot to close said cut in said associated boot wherein each of said flaps is configured to retain said associated boot on the leg;
a pair of inserts, each of said inserts being removably positioned within an associated one of said boots wherein each of said inserts is configured to surround the associated leg and a corresponding foot when said boots are worn, each of said inserts having a foot portion and a leg portion, each of said inserts being comprised of a thermally conductive material; and
a pair of heating units, each of said heating units being coupled to an associated one of said inserts, each of said heating units being in thermal communication with said associated insert, each of said heating units heating said associated insert when said heating units is turned on wherein each of said heating units is configured to heat the associated leg when said boots are worn thereby enhancing blood circulation in the associated leg, each of said heating units comprising;
a heating element being positioned within said associated insert, said heating element being in thermal communication with said associated insert, said heating element generating heat when said heating element is turned on to heat said associated insert, said heating element being looped repeatedly to form a plurality of coils, said coils being distributed along said foot portion and said leg portion of said associated insert;
a control being coupled to said associated insert, said control being electrically coupled to said heating element such that said control turns said heating element on and off, said control actuating said heating element between a minimum temperature, a middle temperature and a maximum temperature; and
a power supply being coupled to said control, said power supply being in electrical communication with said control, said power supply comprising:
at least one battery; and
a charge port being coupled to said control, said charge port being electrically coupled to said at least one battery, said charge port being configured to be electrically coupled to a power source for charging said at least one battery.
